(19) United States
(12) Patent Application Publication
(10) Pub. No.: US 2010/0317639 A1
(43) Pub. Date: Dec. 16, 2010

(54) Medicament Based On a Monoester of Steroids With Long Chain Fatty Acids

(76) Inventor: Anna Petroni, Segrate (IT)

Correspondence Address: Thomas E. Spath;Abelman, Frayne & Schwab, 666 Third Avenue, 10th Floor, New York, NY, 10017-5621, US

(21) Appl. No.: 12/086,673

(22) PCT Filed: Dec. 12, 2006
(86) PCT No.: PCT/EP2006/011925

§ 371 Date: Jun. 12, 2008

(30) Foreign Application Priority Data
Dec. 13, 2005 (IT) MI2005A002377

(51) Int. Cl. A61K 31/56; A61P 25/28; C07J 1/00; A61P 9/10

(52) U.S. Cl. 514/182; 552/650

(57) ABSTRACT

A monoester of a family of steroids with a C₁₆-C₂₄ fatty acid for use as a medicament is disclosed. Particularly the monoester of 5α-androstan-3α,17β-diol with a C₁₆-C₂₄ fatty acid is disclosed, such as preferably linoleic acid in all its isomeric forms. The medicament is capable to reduce the accumulation of cholesterol esters, reducing the ACAT enzyme (acyl-coenzyme A: cholesterol acetyltransferase) effect so that monoesters are used for the manufacture of a medicament for treating adrenoleucodistrophy, Alzheimer's disease and arteriosclerosis.

The present invention concerns a monoester of steroids with either a saturated or an unsaturated C₁₆-C₂₄ fatty acid for use as a medicament. Particularly the present invention is related to a medicament capable to reduce the accumulation of cholesterol esters, by reducing the ACAT enzyme (acyl-coenzyme A: cholesterol acetyltransferase) effect. Advantageously the invention is also related to a new monoester of the androgen, 5α-androstan-3α,17β-diol (briefly named 3α-diol), or the androgen, 5α-androstan-3β,17β-diol (briefly named 3β-diol), with either a saturated or an unsaturated C₁₆-C₂₄ fatty acid.

The ACAT enzyme is responsible for the intracellular esterification of cholesterol. The intracellular accumulation of cholesterol esters, particularly cholesterol esters with saturated fatty acids is considered to be a characteristic sign of arteriosclerosis (“CL277,082: A novel inhibitor of ACAT-catalyzed cholesterol esterification and cholesterol absorption” Journal of Lipid Research, volume 30, 1989). Recently an association between the ACAT enzyme and Alzheimer's disease has been also suggested, because an effect of cholesterol metabolism on amyloid plaques development has been supposed (New way of controlling cholesterol may help treat Alzheimer, Massachusetts General Hospital, published in Oct. 14, 2004).

The inclusion of cholesterol esterified with very long chain saturated fatty acids in the adrenal gland cortex and in testicles is also reported as evidence of the presence of a disease called adrenoleucodistrophy. Specifically adrenoleucodistrophy is a serious hereditary chromosome X-linked disease, by which there is an abnormal accumulation of very long chain saturated fatty acids in plasma and tissues, due to a reduced peroxisomal beta-oxidation.

Metabolic pathways of lipid and steroid metabolism are extremely complex and often correlated, by way of illustration, all steroid hormones, in humans, derive from cholesterol (“Williams textbook of Endocrinology”, IX edition, chapter 12, page 521, Wilson, Foster, Kronenberg, Larsen publishers).

The alteration of lipid and steroid mechanism, with increased concentration of cholesterol esters, appears therefore an evidence for the indicated pathologies. Therefore a use of selective inhibitors for ACAT enzyme has been suggested in order to control the accumulation of cellular and circulating cholesterol esters (“The pharmacological basis of therapeutics”, Goodman and Gilman's, edition X, chapter 36).

In regard to artheriosclerotic plaques, the ACAT inhibitor CL 277,082 (N-(2,4-difluorophenyl)-N-(4-neopentylbenzyl)-N-(n-heptyl)urea) turned out to be very strong and specific in inhibiting the ACAT activity (“Association between acyl-coenzyme A: cholesterol acyltrasferase gene and risk for Alzheimer's disease in Chinese”, Zhao F G. Et al, Department of Neurology, First hospital of Peking University, Neuroscience Lett, Jul. 22, 2005; “On the mechanism by which an ACAT inhibitor (CL 277,082) influences plasma lipoproteins in the rat” Balasubramaniam S et al, Artherosclerosis, 1990 May; 82 (1-2): 1-5).

In order to treat adrenoleucodistrophy, dihydrotestosterone and the androgen 5α-androstan-3α,17β-diol (3α-diol) have been proposed as agents for controlling the metabolism of long chain saturated fatty acids (VLCFA), being capable to increase beta-oxidation and to reduce cholesterol esterification processes in patients suffering from the disease (“Effects of the testosterone metabolite dihydrotestosterone and 5α-androstan-3α,17β-diol on very long chain fatty acid metabolism in X-adrenoleukodystrophic fibroblasts” A. Petroni et al, Life Sciences 73 (2003) 1567-1575 Ed. Elsevier).

Such a study has not provided a potential pharmacological therapy. The androgens as such have been used in the therapy of adrenoleucodistrophy but they didn't have a beneficial effect as reported by Maris (“X-linked adrenoleukodystrophy presenting as neurologically pure familial spastic paraparesis” T. Maris et al, Neurology 45 (1995) 1101-1104).

In order to treat adrenoleucodistrophy a use of erucic acid (C22:1), which is administered in form of triglyceride, has been also proposed (“Dietary Management of X-linked adrenoleukodystrophy” Hugo W. Moser et al, Annual Review of Nutrition, Vol. 15: 379-397, publication date July 1995). Such an acid resulted to be only partially effective in vitro and little effective in vivo because it failed to cross the hematoencephalic barrier.

It has been surprisingly found that some esters of steroids with either saturated or unsaturated C₁₆-C₂₄ fatty acids are capable to intervene in alteration of the lipid and steroid mechanisms, particularly by reducing the accumulation of cholesterol esters.

An object of the present invention is therefore to provide a medicament which is capable to treat diseases that show abnormal accumulation of cellular and circulating cholesterol esters.

An object of the present invention is therefore to provide also a medicament for the treatment of adrenoleucodistrophy, Alzheimer's disease and the treatment of arteriosclerosis.

Such objects have been achieved by providing a family of monoesters for use as a medicament as recited in claim 1, specifically in any one of claims from 2 to 5.

Specifically the present invention is related to a medicament based on a monoester of a steroid of formula I

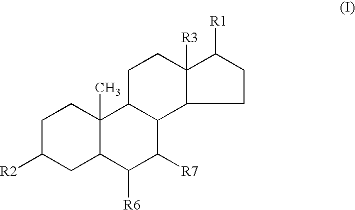

wherein
R1 and R2 are independently —OH or ═O;

### R3 is —CH₃;

R6 and R7 are independently —H or —OH;
or a steroid of formula II

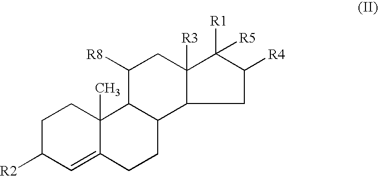

wherein
R1 is selected from the group consisting of ═O, —OH, —COCH₃, and —COCH₂OH;

### R2 is ═O;

R3 and R4 are independently —H, —CH₃, —OH, —CH₂OH or —CHO;
R5 is optionally present and it is —H or —OH;

### R8 is —H or —OH;

or a steroid of formula III

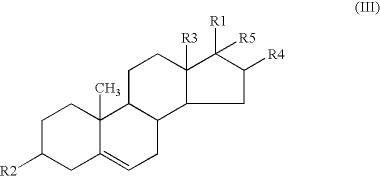

wherein
R1 is selected from the group consisting of ═O and —COCH₃,

### R2 is —OH;

R3 and R4 are independently —H, —CH₃ or —OH;
R5 is optionally present and it is —H or —OH;
or a steroid of formula IV:

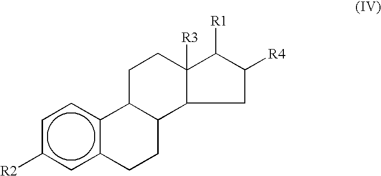

wherein
R1 is selected from the group consisting of —OH and ═O;

### R2 is —OH;

R3 and R4 are independently —H, —CH₃ or —OH;
with either a saturated or an unsaturated C₁₆-C₂₄ fatty acid.

Advantageously the invention concerns a monoester of a steroid having formulas I, II, III or IV with a C₁₈-C₂₂ fatty acid, preferably selected from the group consisting of palmitic acid, palmitoleic acid, oleic acid, stearic acid, elaidic acid, vaccenic acid, linoleic acid, conjugated linoleic acid, linolenic acid, α-linolenic acid, γ-linolenic acid, di-homo-gamma linolenic acid, eleostearic acid, arachidonic acid (eicosatetraenoic acid), adrenic acid, erucic acid, nervonic acid, docosapentaenoic acid, eicosatetraenoic acid, eicosapentaenoic acid as a medicament.

Preferably the fatty acid is an acid selected from the group consisting of linoleic acid, conjugated linoleic acid, linolenic acid, arachidonic acid and isomers and derivatives thereof. More preferably it is linoleic acid (cis,cis-9,12-octadecadienoic acid) (18:2, n-6) or an isomer of conjugated linoleic acid. Among the isomers of conjugated linoleic acid (CLA), linoleic acid c9t11, namely cis,trans-9,11-octadecadienoic acid and linoleic acid t10c12, namely trans,cis-10,12-octadecadienoic acid may be mentioned. Among the derivatives of conjugated linoleic acid, cis,cis,trans-6,9,11-octadecatrienoic acid (18:3), cis,cis,trans-8,11,13-eicosatrienoic acid (20:3), cis,cis,trans-8,11,13-eicosatetraenoic acid (20:4) may be mentioned.

Among the derivatives of linolenic acid, gamma-linolenic acid (cis,cis,cis-6,9,12-octadecatrienoic acid) may be mentioned, among the derivatives of alfa-linolenic acid (cis,cis,cis-9,12,15-octadecatrienoic acid), eicosapentaenoic acid (cis,cis,cis,cis-5,8,11,14,17-eicosapentaenoic acid) may be mentioned.

The steroid of formula I will be preferably selected from the group consisting of 5α-androstan-17β-ol-3-one (DHT), 5α-androstan-3α,17β-diol (3α-diol), 5α-androstan-3β,17β-diol (3β-diol), 5α-androstan-3β,6α,17β-triol (6α-triol), 5α-androstan-3β,7β,17β-triol (7β-triol), 5α-androstan-3β,7α,17β-triol (7α-triol), 5α-androstan-3,17-dione (androstandione), 5α-androstan-3α-ol-17-one (androsterone); the steroid of formula II will be preferably selected from the group consisting of testosterone, androstenedione, 16α-hydroxyandrostenedione, progesterone, 17α-hydroxyprogesterone, deoxycorticosterone, 11-deoxycortisol, cortisol, corticosterone, 18-hydroxycorticosterone, aldosterone; the steroid of formula III will be preferably selected from the group consisting of dehydroepiandrosterone, 17α-hydroxypregnenolone, pregnenolone; and the steroid of formula IV will be preferably selected from the group consisting of estrone, estradiol, estriol.

In a preferred and advantageous embodiment the monoester is composed by a steroid of formula IV, also known as androgen.

In another aspect the invention concerns a new monoester of a steroid of formula V

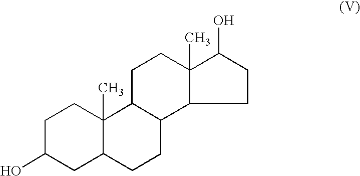

with a C₁₆-C₂₄ fatty acid. Preferably, the steroid of Formula V is 5α-androstan-3α,17β-diol (3α-diol) or 5α-androstan-3β,17β-diol (3β-diol).

Such a monoester with the androgen 3α-diol or 3β-diol will be preferably an ester composed by a fatty acid selected from the group consisting of linoleic acid, conjugated linoleic acid and derivatives thereof, linolenic acid, arachidonic acid and isomers thereof, even more preferably it is an ester with linoleic acid (cis,cis-9,12-octadecadienoic acid (18:2, n-6)).

Preferably, the monoester of the invention is formed between the steroid and the fatty acid at either the position 3 or the position 17 of the steroid, the structure of which is usually numbered in the art as follows:

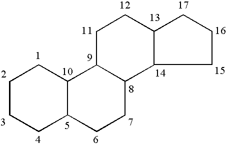

The monoester family of the invention may be used for the manufacture of a medicament for reducing the accumulation of cellular and circulating cholesterol esters as recited in claim 22.

Without wishing to be bound by any theory, as the ACAT enzyme plays a key role in the metabolism of fatty acids by forming cholesterol esters, it is believed that the reduction of the accumulation of cellular and circulating cholesterol esters can be achieved by reducing the activity of ACAT enzyme.

Thus, the medicament according to the invention is capable to perform its action in pathologies, in which an accumulation of cholesterol esters is seen and in which the ACAT enzyme plays a key role, such as adrenoleucodistrophy, Alzheimer's disease and arteriosclerosis.

Monoesters according to the invention may be prepared by organic chemistry synthesis methods, well known to those skilled in the art, which include the esterification reaction of the steroid with the carboxy group of the fatty acid.

Monoesters according to the invention may be also added with a pharmaceutically acceptable carrier for the manufacture of pharmaceutical compositions.

The invention will be now disclosed with reference to some examples of biochemical preparation and to an example of effectiveness on the inhibition of ACAT enzyme. Such examples are given by way of illustration of the invention, and are not to be considered limiting the invention in any way.

### EXAMPLE 1

### Preparation of the monoester of the androgen 5α-androstan-3α,17β-diol (3α-diol) and of the linoleic acid (LA) (cis,cis-9,12-octadecadienoic acid) (18:2, n-6)

For the preparation of the ester according to the invention, rat liver microsomes were used, which have an enzymatic system enabling the esterification of the carboxy group of fatty acids (in this case LA) with —OH group in 3 of steroids, in this case 3α-diol.

Preparation of Rat Liver Microsomes

Microsomes were obtained by following the method by Einarsson et al., 1989.

The following buffers were prepared separately:

Tris-HCl buffer: pH 7.4, 50 mM, 500 ml with 50 mM Tris buffer, 0.3 M Sucrose, 1 mM EDTANa₂ and protease inhibitors (PMSF phenyl methyl sulphonyl fluoride (1 mM) and leupeptin (50 micromolar))

Potassium phosphate buffer: 0.1 M of potassium phosphate buffer pH 7.4, volume: 500 ml with 1 mM EDTA.

Rat liver was taken and weighed (9.56 g), then perfused with physiological saline. “Potter” (glass system with pestle) was used to allow a gentle homogenation of the product obtained from perfusion with 50 ml of Tris-HCl buffer, pH 7.4.

Therefore, the solution was centrifuged at 20,000 revolutions for 15 min at 4° C. Then the surnatant was taken and centrifuged with ultracentrifuge at 100,000 revolutions for 1 hour (40,000 rpm).

The obtained pellet, consisting of microsomes, was then resuspended in 5 ml of potassium phosphate buffer, and subdivided in aliquots, that were freezed at −20° C., to be used in the different experiments.

One of these aliquots was used for protein assay in order to have a valid quantitative reference parameter in the different experiments. A volume of 250 microliters was used to which 500 microliters of soda were added. The solution was left to room temperature and the assay was performed according to classic procedure of Lowry et al., 1951.

The aliquot showed to have a protein concentration of 13.2 micrograms/microliter.

### Synthesis of the ester of 3α-diol with linoleic acid (LA) (cis,cis-9,12-octadecadienoic acid) (18:2, n-6).

Synthesis of the ester was performed in duplicate. Two samples were then prepared (SAMPLE 1 and SAMPLE 2). The two samples were incubated with equal reagents in the same experimental conditions in order to compare the method reproducibility. Each sample contained 5 microliters of said microsomal preparation corresponding to 66 micrograms of proteins.

The solution A was prepared:

MOPS (400 mM), MgCl₂ (50 mM), KCl (50 mM), CoASH (1 mM), ATP (100 mM), α-cyclodextrin (1 mg in 50 microliters H₂O per sample) and biphosphate buffer (42 microliters/sample).

In the two samples H₂O was added in order to have a final volume of 500 microliters per sample.

The following labelled compound was used:

androstan-3α,17β-diol,5α-(9, 11-3H(N))(3α-diol-3H) (mother liquor)

conc. 1 mCi/ml in ethanol

specific activity 40 Ci/mmol

supplied by PerkinElmer Life Sciences, Inc.

In the two above samples the following:

linoleic acid: 72 microM (10.1 micrograms, 0.036 micromoles/sample)

3α-diol-3H: 0.1 microM (2 microCi/2 microliters)

3α-diol: 72 microM (10.1 micrograms, 0.036 micromoles/sample)

were incubated respectively.

The samples have been therefore incubated in said solution A for 10 min at 37° C.

The incubation was therefore blocked for both samples with 2.5 ml of Dole's reagent (consisting of isopropilic alcohol 40 ml, heptane 10 ml, 1N H₂SO₄1 ml), 0.9 ml of H₂O, 1.5 ml of heptane.

After stirring, the separation in two phases was achieved. The upper phase was transferred in a tube. The lower phase was extracted twice more with 1.5 ml of heptane and the two upper phases obtained were joined with the first one in a single tube. The content was dried and taken-up with 1 ml of chloroform:methanol 2:1.

An aliquot per sample was then taken which was used for the radiation count with scintillation liquid (Ultima Gold).

The count was performed according to classic methods as reported in “Radioisotopes in Biology, a practical approach”, published by R. J. Slater, Oxford University Press, 1990; “Effects of simvastatin on the metabolism of polyunsaturated fatty acids and on glycerolipid, cholesterol and de novo lipid synthesis in THP-1 cells” Risè et al. Journal Lipid Research, volume 38, 1299-1307, 1997.

On the basis of the obtained radioactivity, the following ester concentration of LA with 3α-diol resulted:

SAMPLE 1: 15.17 microM (0.015166 micromoles, 8.38 micrograms)

SAMPLE 2: 15.02 microM (0.015016 micromoles, 8.33 micrograms)

### EXAMPLE 2

### Preparation of the ester of the 3α-diol with conjugated linoleic acid (CLA), isomer C9t11

The example 1 was repeated in order to obtain the monoester of the 3α-diol with the isomer of the linoleic acid (c9t11) starting from:

conjugated linoleic acid: 90 microM

3α-diol-3H: 0.1 microM (2 microCi/2 microliters)

3α-diol: 90 microM

The following results were obtained:

Sample 1: 18.01 microM

Sample 2: 19.6 microM

### EXAMPLE 3

### Preparation of the monoester of the androgen 5α-androstan-3α,17β-diol (3α-diol) and of the linoleic acid (LA) (cis,cis-9,12-octadecadienoic acid) (18:2, n-6) by means of double labelling

Rat liver microsomes obtained according to the method disclosed in example 1 were used.

The synthesis was performed in two steps.

In the first step the linoleic acid (cis,cis-9,12-octadecadienoic acid) (18:2, n-6) was incubated in 60 micrograms of microsomes.

Specifically a solution containing MOPS (400 mM), MgCl₂ (50 mM), KCl (50 mM), CoASH (1 mM), ATP (100 mM), α-cyclodextrin (1 mg in 50 microliters H₂O per sample) and biphosphate buffer (42 microliters/sample) was prepared.

In the sample of microsomes, H₂O was added in order to have a final volume of 500 microliters. Therefore linoleic acid (C18:0) 72 microM (0.036 micromolar) was incubated with 2 microCi of ¹⁴C-labelled linoleic acid (specific activity 55 mCi/mmoles, 5.5 microCi/0.1 micromol Amersham, Bioscience, UK).

Then incubation for 10 min at 37° C. and subsequent extraction with Dole's reagent have been performed as disclosed in example 1. The extract was dried and taken-up again in liquid. An aliquot of liquid was counted in a beta counter.

In the second step of the preparation, said extract was taken-up again in 1 ml of water, to which BSA 1 mg/ml in phosphate buffer, EDTA (200 microliters), microsomes 60 micrograms, 3α-diol-3H (0.1 microM (2 microCi/2 microliters), 3α-diol 72 microM (10.1 micrograms, 0.036 micromoles/sample) were added. EDTA phosphate buffer was also added in order to have a final volume of 2000 microliters. The mixture has been incubated for 5 min at 37° C. An aliquot was then subjected to TLC with use of a standard in order to separate the ester and the ester-related band was transferred in a tube by extraction and subjected to radiation count in double labelling (labelling of the androgen and of the linoleic acid). Separate reading of two isotopes was performed: respectively ³H for 3α-diol and ¹⁴C for linoleic acid. The reading was automatically done by the beta counter instrument. The count was done according to classic methods as reported in “Radioisotopes in Biology, a practical approach”, published by R. J. Slater, Oxford University Press, 1990; “Effects of simvastatin on the metabolism of polyunsaturated fatty acids and on glycerolipid, cholesterol, and de novo lipid synthesis in THP-1 cells” Risè et al. Journal Lipid Research, volume 38, 1299-1307, 1997.

On the basis of obtained radioactivity, the following ester concentration resulted: 4.5 microM.

### EXAMPLE 4

### Preparation of the monoester of the androgen 5α-androstan-3α,17β-diol (3α-diol) and of the linoleic acid (LA) (cis,cis-9,12-octadecadienoic acid) (18:2, n-6) with execution of purification step

The example 1 was repeated both for the preparation of rat liver microsomes and the synthesis of the ester until the aliquots were taken-up for the radiation count with scintillation liquid (Ultima Gold).

Before performing the count, the ester-containing aliquots were purified using thin layer chromatography (TLC).

Two silica gel 60 TLC plates (20×20 cm) (Merck) were used for each aliquot, taken-up from the samples respectively. The plates have previously been heat-activated at 120° C. for 30 min, to remove dampness, which would have influenced the run of the compounds with TLC during different periods of the year. The plates were also cooled in an air-free glass system before use.

The two samples were run on the two plates using the following solvents: hexane:diethylether:acetic acid in amounts equivalent to 70:30:1.5.

The samples were run in the same chromatographic conditions, by using linoleic acid and 3α-diol as standards, which served to verify the reproducibility of the chromatography.

At the end of the run the plates were subjected to a nitrogen flow in order to remove the solvents and highlight the standard-corresponding bands and those produced by the two samples. The bands similar to standard bands resulted to be the reagents, linoleic acid and androstan, and the remaining to the formed ester. The silica corresponding to the ester bands of the two plates was then taken-up and put in two tubes respectively. The content of each tube corresponded to one band, which was extracted with the following solvents: methanol 5 ml, chloroform 2.5 ml. After stirring in vortex, further 2.5 ml of chloroform were used for the extraction, to which a further stirring followed and then an addition of 2.5 ml of H₂O, for 2 hours at −20° C. in order to separate the phases. Therefore, the lower phases of the extraction from each tube were kept, which were dried and resuspended separately with 1 ml of chloroform:methanol (2:1). An aliquot per sample was then taken-up and it was subjected to count by beta counter.

On the basis of obtained radioactivity, the following LA ester with 3α-diol concentration resulted:

SAMPLE 1: 2.98 microM (0.00297 micromoles, 1.65 micrograms)

SAMPLE 2: 3.64 microM (0.00364 micromoles, 2.01 micrograms)

### EXAMPLE 5

### Identification by Mass Spectrometry

The esters of linoleic acid (LA) (C18:2, n-6) with the androgen 5α-androstan-3α,17β-diol (3α-diol) obtained with the synthesis method of the example 1 were analyzed by mass spectrometry for quantitative determination of the formed ester.

The instrument Massa quadrupolo LC massa, (Waters) 4 Ultima Platinum was used.

The used source was an electronspray source, positive and negative ions.

The two samples obtained from example 1 were dried in nitrogen flow and taken-up again in 500 microliters of acetonitrile; the injection volume was 200 microliters. Tuning for standards was performed: cholesteryl linoleate and 3α-diol (1-10 micrograms/ml).

On the basis of the molecular peak corresponding to molecular weight (555) it has been confirmed that the two samples obtained from example 1 resulted to be the ester of 3α-diol with linoleic acid.

### EXAMPLE 6

### Evaluation of Effectiveness of Ester According to the Invention

For the experiments of effectiveness a test to evaluate the effect of the ester formed from linoleic acid obtained from example 1 and example 4 on acyl-coenzyme A:cholesterol acetyltransferase enzyme (ACAT) activity was performed. The experiment was performed in vitro according to a classic pharmacology method, used for the evaluation of the activity of this enzyme, reported in the method book: Drug discovery and evaluation, Pharmacological assay, Ed. H. G: Vogel (Springer), p. 1119, 2002.

Hepatic microsomes were prepared as well as in the example 1.

Eight tubes were prepared, in all tubes linoleic acid-¹⁴C (10 microM) was incubated in presence of 200 micrograms of microsomes. Each tube contained phosphate buffer 0.04 M KH₂PO₄, pH 7.4 with 0.05 M KCl, 0.03 EDTA, 0.3 M sucrose, 1 mM α-cyclodextrin, final volume 750 microliters. The eight tubes have been incubated at 37° C. for 10 min.

The esters of example 1 and 4 were pooled together and three sample solutions of concentration 12.5, 25 and 50 micromolar respectively were prepared in duplicate, therefore obtaining six sample solutions.

The six sample solutions were then transferred respectively in six of eight tubes prepared as above at the end of the 10 minutes (each concentration of ester separately incubated per tube). The seventh tube was added with ¹⁴C-linoleic acid alone (without esters) and the eighth with ethanol alone (carrier in which the esters were resuspended) to evaluate a possible effect of ethanol on the enzymatic activity. All the eight tubes have been incubated at 37° C. for four minutes.

At the end of the time the reaction was stopped by adding chloroform:methanol 2:1. The samples of the eight tubes were extracted according to the Folch's method (1957). The lower phase of each sample containing the lipid extracts was separated and dried, then taken-up again in 1 ml of chloroform:methanol 2:1. The extract of each sample was dried and subjected to TLC.

A preparation of cholesteryl oleate and cholesteryl palmitoleate which run together in TLC was used as a standard and hexane:diethyleter:acetic acid 70:30:1.5 were used as solvents.

Spots were highlighted through iodine vapour. The bands which correspond to the standard for each TLC were taken off from the plate and counted in beta counter.

The obtained results (cpm) per sample were transformed in dpm/mg proteins and in inhibition percentage with reference to the untreated sample. The sample constituted by carrier alone resulted non-effective. An inhibition of 44%+/−3.6 (mean+/−standard error) for the 25 microM concentration resulted, the effect resulted to be lower at concentrations 50 and 12.5 microM that is, 28%+/−1.9 and 2.5+/−2, respectively.

As evident from the above example, monoesters according to the invention are capable to reduce the accumulation of cholesterol esters by reducing the ACAT enzyme effect and indirectly intervene on β-oxidation in peroxisomes, a mechanism involved in the accumulation of very long chain saturated fatty acids, which are responsible for severe clinical symptoms, particularly for adrenoleucodistrophy. Thus, the use of the medicament according to the invention is extremely advantageous for treatment of pathologies involving a lipid metabolism alteration, for instance adrenoleucodistrophy, Alzheimer's disease and treatment of arteriosclerosis.

Although the invention has been disclosed with reference to some embodiments and the effectiveness of particular steroids and acids, the use of other steroids and fatty acids may be provided by the skilled person in the art for the preparation and use of the medicament according to the invention, without however departing from the scope defined by the appended claims.

1. A monoester of a steroid selected from the group consisting of:
(i) a steroid of formula I: 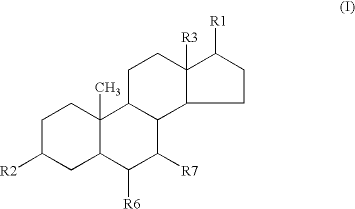
wherein
R1 and R2 are independently —OH or ═O;
R3 is —CH₃;
R6 and R7 are independently —H or —OH;
(ii) a steroid of formula II 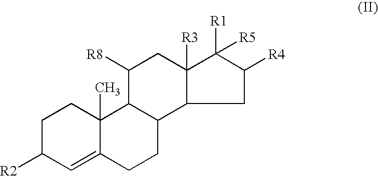
wherein
R1 is selected from the group consisting of ═O, —OH, —COCH₃, and —COCH₂OH;
R2 is ═O;
R3 and R4 are independently —H, —CH₃, —OH, —CH₂OH or —CHO;
R5 is optionally present and is —H or —OH;
R8 is —H or —OH;
(iii) a steroid of formula III 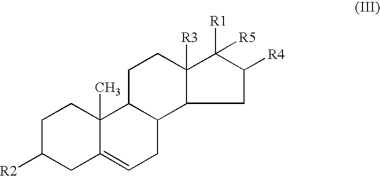
wherein
R1 is selected from the group consisting of ═O and —COCH₃,
R2 is —OH;
R3 and R4 are independently —H, —CH₃ or —OH;
R5 is optionally present and is —H or —OH; and
(iv) a steroid of formula IV 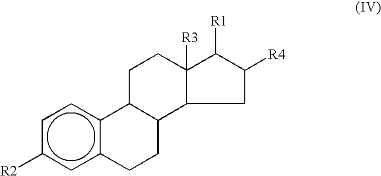
wherein
R1 is selected from the group consisting of —OH and ═O;
R2 is —OH;
R3 and R4 are independently —H, —CH₃ or —OH;
with either a saturated or an unsaturated C₁₆-C₂₄ fatty acid
for use as a medicament.

2. The monoester of claim 1 which is a monoester of a steroid of formula I: 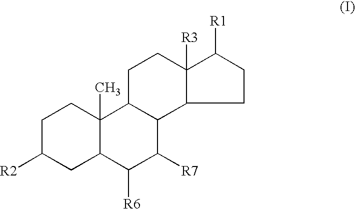
wherein
R1 and R2 are independently —OH or ═O;
R3 is —CH₃;
R6 and R7 are independently —H or —OH;
with either a saturated or an unsaturated C₁₆-C₂₄ fatty acid
for use as a medicament.

3. The monoester of claim 1 which is a monoester of a steroid of formula II 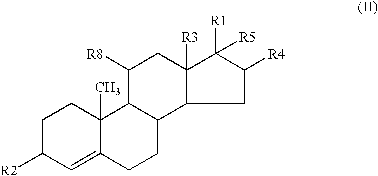
wherein
R1 is selected from the group consisting of ═O, —OH, —COCH₃, and —COCH₂OH;
R2 is ═O;
R3 and R4 are independently —H, —CH₃, —OH, —CH₂OH or —CHO;
R5 is optionally present and is —H or —OH;
R8 is —H or —OH;
with either a saturated or an unsaturated C₁₆-C₂₄ fatty acid,
for use as a medicament.

4. The monoester of claim 1 which is a monoester of a steroid of formula III 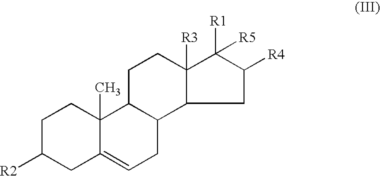
wherein
R1 is selected from the group consisting of ═O and —COCH₃,
R2 is —OH;
R3 and R4 are independently —H, —CH₃ or —OH;
R5 is optionally present and is —H or —OH;
with either a saturated or an unsaturated C₁₆-C₂₄ fatty acid,
for use as a medicament.

5. The monoester of claim 1 which is a monoester of a steroid of formula IV 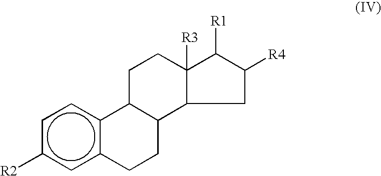
wherein
R1 is selected from the group consisting of —OH and ═O;
R2 is —OH;
R3 and R4 are independently —H, —CH₃ or —OH;
with either a saturated or an unsaturated C₁₆-C₂₄ fatty acid,
for use as a medicament.

6. The monoester according to any one of claims 1 to 5 wherein the monoester is formed between the steroid and the fatty acid at either the position 3 or the position 17 of steroid of formula I, II, III or IV.

7. The monoester according to any one of claims 1 to 6, wherein the saturated or unsaturated fatty acid is a C₁₈-C₂₂ fatty acid.

8. The monoester according to claim 7, wherein the fatty acid is selected from the group consisting of palmitic acid, palmitoleic acid, oleic acid, stearic acid, elaidic acid, vaccenic acid, linoleic acid, conjugated linoleic acid and derivatives thereof, linolenic acid, α-linolenic acid, γ-linolenic acid, homo-gamma-linoleic acid, eleostearic acid, arachidonic acid, adrenic acid, erucic acid, nervonic acid, docosapentaenoic acid, eicosatetraenoic acid, eicosapentaenoic acid.

9. The monoester according to claim 8, wherein the fatty acid is an acid selected from the group consisting of palmitic acid, linoleic acid, conjugated linoleic acid and derivatives thereof, linolenic acid, arachidonic acid and isomers thereof.

10. The monoester according to claim 9, wherein the fatty acid is linoleic acid or conjugated linoleic acid.

11. The monoester according to any one of claims 1, 2, 6-10 wherein the steroid of formula I is selected from the group consisting of 5α-androstan-17β-ol-3-one (DHT), 5α-androstan-3α,17β-diol (3α-diol), 5α-androstan-3β,17β-diol (3β-diol), 5α-androstan-3β,6α,17β-triol (6α-triol), 5α-androstan-3β,7β,17β-triol (7β-triol), 5α-androstan-3β,7α,17β-triol (7α-triol), 5α-androstan-3,17-dione (androstandione) and 5α-androstan-3α-ol-17-one (androsterone).

12. The monoester according to any one of claims 1, 3, 6-10 wherein the steroid of formula II is selected from the group consisting of testosterone, androstenedione, 16α-hydroxyandrostenedione, progesterone, 17α-hydroxyprogesterone, deoxycorticosterone, 11-deoxycortisol, cortisol, corticosterone, 18-hydroxycorticosterone, aldosterone.

13. The monoester according to any one of claims 1, 4, 6-10 wherein the steroid of formula III is selected from the group consisting of dehydroepiandrosterone, 17α-hydroxypregnenolone and pregnenolone.

14. The monoester according to any one of claims 1, 5, 6-10, wherein the steroid of formula IV is selected from the group consisting of estrone, estradiol, estriol.

15. The monoester of claim 11 wherein the steroid of formula I is 5α-androstan-3α,17β-diol (3α-diol).

16. A monoester of a steroid of formula V: 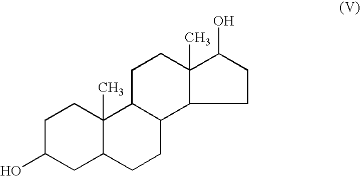
with a C₁₆-C₂₄ fatty acid.

17. The monoester according to claim 16, wherein the steroid is 5α-androstan-3α,17β-diol (3α-diol) or 5α-androstan-3β,17β-diol (3β-diol).

18. The monoester according to claim 16 or claim 17, wherein the fatty acid is selected from the group consisting of linoleic acid, conjugated linoleic acid and derivatives thereof, linolenic acid, arachidonic acid and isomers thereof.

19. The monoester according to claim 18, wherein the fatty acid is linoleic acid (cis,cis-9,12-octadecadienoic).

20. The monoester according to any one of claims 16 to 19 for use as a medicament.

21. A pharmaceutical composition comprising one or more monoesters according to any one of claims 1 to 15 and a pharmaceutically acceptable carrier.

22. Use of a monoester according to any of claims 1 to 15 or according to any of claims 16 to 19 for the manufacture of a medicament for reducing the accumulation of cellular and circulating cholesterol esters.

23. Use according to claim 22, wherein the reduction of the accumulation of cholesterol esters occurs in the treatment of adrenoleucodistrophy.

24. Use according to claim 22, wherein the reduction of the accumulation of cholesterol esters occurs in the treatment of Alzheimer's disease.

25. Use according to claim 22, wherein the reduction of the accumulation of cholesterol esters occurs in the treatment of arteriosclerosis.
